SEQUENCE LISTING (5 sequences)

SEQ ID NO: 1 - protein, 593 aa, Homo sapiens
MWTLVSWVALTAGLVAGTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 2 - DNA, 20 bp, Artificial Sequence
  forward primer for PGRN
ttcacacacgatgcgtttca

SEQ ID NO: 3 - DNA, 20 bp, Artificial Sequence
  reverse primer for PGRN
agggcacacgacagaaaaag

SEQ ID NO: 4 - DNA, 20 bp, Artificial Sequence
  forward primer for GAPDH
accacagtccatgccatcac

SEQ ID NO: 5 - DNA, 20 bp, Artificial Sequence
  reverse primer for GAPDH
tccaccaccctgttgctgta

(19) United States
(12) Patent Application Publication
(10) Pub. No.: US 2016/0319286 A1
(43) Pub. Date: Nov. 3, 2016

(54) COMPOSITION FOR PREVENTING OR TREATING OSTEOPOROSIS, CONTAINING PROGRANULIN INHIBITOR AS ACTIVE INGREDIENT

(71) Applicant: OSTEONEUROGEN, Seongnam-si Gyeonggi-do (KR)

(72) Inventors: Byung Soo YOUN, Gyeonggi-do (KR); Jae Min OH, Jeollabuk-do (KR); Ju Young KIM, Jeollabuk-do (KR); Myeung Su LEE, Jeollabuk-do (KR)

(73) Assignee: OSTEONEUROGEN, Gyeonggi-do (KR)

(21) Appl. No.: 15/029,630

(22) PCT Filed: Oct. 15, 2014
(86) PCT No.: PCT/KR2014/009664

§ 371 Date: Jul. 21, 2016

(30) Foreign Application Priority Data
Oct. 15, 2013 (KR) 10-2013-0122723

(51) Int. Cl. C12N 15/113; G01N 33/68; C12Q 1/68

(52) U.S. Cl. CPC C12N 15/1136; C12Q 1/6883; G01N 33/6893; C12Q 2600/158; C12Q 2600/136; C12Q 2600/118; G01N 2800/52; G01N 2800/108; G01N 2500/04; G01N 2500/10; C12N 2310/14; C12N 2310/531

(57) ABSTRACT

Disclosed is a composition for inducing the differentiation of osteoclasts containing progranulin as an active ingredient, or a composition for the prevention or treatment of osteoporosis containing a progranulin inhibitor as an active ingredient, wherein progranulin functions as an RANK-dependent cell-communication factor and as a mediator in osteoporosis through inflammation, and exists at a remarkably high concentration, compared to a control, in the serum of prostate cancer patients, osteoporosis patients, and bedridden patients with low bone mineral density, whereby the progranulin can be used as an osteoclast differentiation factor and as a biomarker for osteoporosis or metastatic prostate cancer.

### TECHNICAL FIELD

The present invention relates to a composition for inducing the differentiation of osteoclasts, containing progranulin as an active ingredient, or a composition for the prevention or treatment of osteoporosis, containing a progranulin inhibitor as an active ingredient.

### BACKGROUND ART

Human bones function as a rigid scaffold resulting from the differentiation of osteoblasts and osteoclasts and balance of the quantities thereof, as well as the accumulation of minerals. The quantitative reduction of estrogen after menopause in women is known to promote the differentiation of osteoclasts to thus decrease bone mineral density, leading to osteoporosis. This mechanism is replicable in animal models in vivo and in vitro.

Individual nuclei of mature osteoclasts are changed into coenocytes through cell fusion, and a bundle of cells fused with actin rings breaks existing bone tissues, whereby osteoporosis arises. The progenitor cells of osteoclasts belong to the myeloid cell lineage. When bone marrow cells are treated with M-CSF (macrophage-colony stimulating factor), they are differentiated into preosteoclasts and macrophages. When the bone marrow cells are treated with RANK-ligand (RANKL) as the master regulator for the differentiation of osteoclasts, they are coupled with the receptor RANK to thus make a variety of signal transmissions. Therefore, the transfer factor NFAT-c1 is activated through activation of Ca²⁺-dependent kinase due to changes in the Ca-oscillator in the cells, thereby completing the expression of various proteins necessary for the differentiation of osteoclasts. Furthermore, cell-communication (coupling) factors, such as TGF-β, IGF, IFN-γ, TNF-α and SemaD, which are expressed in osteoblasts or osteoclasts to thus adjust the amount and rate of differentiation of osteoclasts, act like RANKL, thus regulating the differentiation and killing of osteoclasts under both homeostatic and pathological conditions.

Progranulin is an 88 kD glycoprotein that consists of seven and a half repeating units of granulin (Grn) domains composed of twelve “cysteine-rich motifs”. For the human proteome atlas, it exists as an 80 kD glycosylated protein in the serum or plasma. Although progranulin is called proepithelin and prostate cancer (PC) cell-derived growth factor in some circles, the official name thereof is “GRN” based on HUGO nomenclature.

The granulin peptide was first discovered as a peptide secreted from leukocytes (Bateman et.al., BBRC, 1990). Granulin has previously been reported to function as a wound-healing factor (Bateman et. al., Nature Medicine, 2003), and also as a neuronal growth factor (Bateman et. al., BMC Neuroscience, 2009; and Van Damme et, al., JCB, 2008). Furthermore, it is reported that granulin is the causal gene of tau-negative familial FTD (Baker & Cruts et. al., Nature, 2006) and acts as a metabolic hormone (Youn et. al., Diabetes, 2009), an appetite-suppressing hormone (Kim et al., Endocrinology, 2011), and an insulin resistance factor (Matsubara et. al., Cell Metabolism, 2012). However, to date there have been no reports associating granulin with the differentiation of osteoclasts or with osteoporosis.

Accordingly, the present inventors have ascertained that progranulin acts as the RANK-dependent cell-communication factor in in-vivo and in-vitro testing with mice, and also as a mediator in osteoporosis through LPS-induced inflammation, and in human beings, progranulin is present at very high concentrations, compared to a control, in the serum of patients who suffer from prostate cancer and osteoporosis, and bedridden patients with low bone mineral density, whereby progranulin is proven to be the osteoclast differentiation factor and is a biomarker for osteoporosis or a serum biomarker for prostate cancer, thus culminating in the present invention.

### DISCLOSURE

### Technical Problem

Therefore, an object of the present invention is to provide the use of progranulin as an osteoclast differentiation factor, and as a biomarker for osteoporosis or a serum biomarker for metastatic prostate cancer.

### Technical Solution

In order to accomplish the above object, the present invention provides a pharmaceutical composition for prevention or treatment of osteoporosis, containing a progranulin inhibitor as an active ingredient.

In addition, the present invention provides a pharmaceutical composition for inhibiting differentiation of osteoclasts, containing a progranulin inhibitor as an active ingredient.

In addition, the present invention provides a method for diagnosis, treatment or prognostic evaluation of osteoporosis, comprising: 1) measuring an mRNA or protein expression level of progranulin in blood, plasma or serum separated from a subject individual; and 2) screening the subject individual in which the mRNA or protein expression level of progranulin is increased compared to a normal control.

In addition, the present invention provides a kit for diagnosis, treatment or prognostic evaluation of osteoporosis, including any one selected from the group consisting of a nucleic acid complementary to a progranulin gene, a primer or probe specific to a progranulin gene, and an antibody binding to a progranulin protein.

In addition, the present invention provides a method of screening an agent for prevention or treatment of osteoporosis, comprising: 1) treating a progranulin expression cell line with a subject composition or compound; 2) measuring an mRNA or protein expression level of progranulin in the cell line; and 3) screening the subject composition or compound in which the mRNA or protein expression level of progranulin is decreased compared to a non-treated control.

In addition, the present invention provides a method of screening an agent for inhibiting differentiation of osteoclasts, comprising: 1) treating a progranulin expression cell line with a subject composition or compound; 2) measuring an mRNA or protein expression level of progranulin in the cell line; and 3) screening the subject composition or compound in which the mRNA or protein expression level of progranulin is decreased compared to a non-treated control.

In addition, the present invention provides a composition for inducing differentiation of osteoclasts, containing progranulin as an active ingredient.

In addition, the present invention provides a pharmaceutical composition for inhibiting bone metastasis of a prostate cancer cell line caused by differentiation of osteoclasts, containing a progranulin inhibitor as an active ingredient.

In addition, the present invention provides a method of measuring an expression level of progranulin for providing information about diagnosis, treatment or prognostic evaluation of bone metastasis of prostate cancer, comprising: 1) measuring an mRNA or protein expression level of progranulin in blood, plasma or serum separated from a subject individual; and 2) screening the subject individual in which the mRNA or protein expression level of progranulin is increased compared to a normal control.

In addition, the present invention provides a kit for diagnosis, treatment or prognostic evaluation of bone metastasis of prostate cancer, including any one selected from the group consisting of a nucleic acid complementary to a progranulin gene, a primer or probe specific to a progranulin gene, and an antibody binding to a progranulin protein.

In addition, the present invention provides a method of screening an agent for inhibiting bone metastasis of a prostate cancer cell line caused by differentiation of osteoclasts, comprising: 1) treating a progranulin expression cell line with a subject composition or compound; 2) measuring an mRNA or protein expression level of progranulin in the cell line; and 3) screening the subject composition or compound in which the mRNA or protein expression level of progranulin is decreased compared to a non-treated control.

### Advantageous Effects

According to the present invention, progranulin acts as a RANK-dependent cell-communication factor or a mediator in osteoporosis through LPS-induced inflammation. Furthermore, progranulin has a high expression level, compared to a control, in the serum of patients who suffer from prostate cancer and osteoporosis and bedridden patients with low bone mineral density, whereby the use of progranulin as a biomarker is effective at developing an agent for inhibiting bone metastasis of prostate cancer cells or an osteoporosis therapeutic agent.

### DESCRIPTION OF DRAWINGS

FIG. 1 illustrates the mechanism associated with the differentiation of osteoclasts;

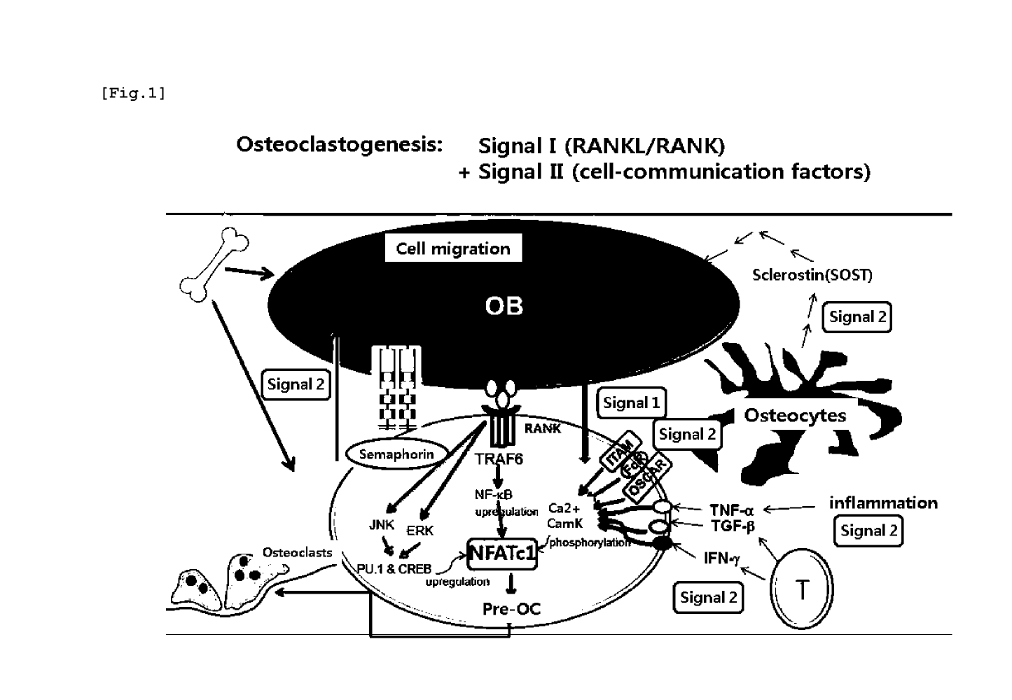

FIG. 2 illustrates the extent of promotion of the differentiation of RANK-dependent osteoclasts depending on the concentration of progranulin in in-vitro testing with mice;

FIG. 3 illustrates the extent of inhibition of the differentiation of RANK-dependent osteoclasts due to progranulin expression knockdown in in-vitro testing with mice;

FIG. 4 is of graphs illustrating the high expression level of progranulin, compared to a normal control, in the serum of osteoporosis mouse models through inflammation in in-vivo testing with mice; and

FIG. 5 is of graphs illustrating the high expression level of progranulin, compared to a normal control, in the serum of human prostate cancer patients, osteoporosis patients, and bedridden patients with low bone mineral density.

### MODE FOR INVENTION

Hereinafter, a detailed description will be given of the present invention.

The present invention addresses a pharmaceutical composition for the prevention or treatment of osteoporosis, containing a progranulin inhibitor as an active ingredient.

Also, the present invention addresses a pharmaceutical composition for inhibiting the differentiation of osteoclasts, containing a progranulin inhibitor as an active ingredient.

The progranulin is preferably composed of the amino acid sequence of SEQ ID NO: 1, in which any one or more amino acids of SEQ ID NO: 1 may be subjected to addition, deletion or substitution, but the present invention is not limited thereto.

The progranulin inhibitor may be an expression or activation inhibitor. Specifically, the expression inhibitor of a progranulin protein is preferably any one selected from the group consisting of an antisense nucleotide, and RNAi [short interfering RNA, short hairpin RNA and micro RNA (miRNA)], each of which complementarily binds to mRNA of a progranulin gene, and the activation inhibitor of a progranulin protein is preferably any one selected from the group consisting of a compound, a peptide, peptide mimetics, an aptamer, and an antibody, each of which complementarily binds to a progranulin protein, but the present invention is not limited thereto.

The specific examples thereof are described below.

1) RNAi

RNA interference (RNAi) is a post-transcriptional gene silencing mechanism that causes the degradation of corresponding mRNA by introducing double-stranded RNA (dsRNA), corresponding to a progranulin gene, into cells or organisms. Due to the RNAi effect, multiple cell division continues before gene expression is restored, and thus, RNAi is very effective at making a knockout or knockdown of interest at the RNA level (Elbashir et al. Nature May 24; 411(6836):494-8, 2001). The RNAi technique is a standard molecular biological method for gene silencing. The dsRNA, corresponding to the sequence of the target gene to be inactivated, may be produced through a standard method, for example, simultaneous transcription of both strands of template DNA using T7 RNA polymerase. Useful as the kit for producing dsRNA used for RNAi may be a commercially available product. The method of transfection of dsRNA or a plasmid processed so as to manufacture dsRNA is a known technique.

2) Antisense Nucleic Acid Sequence

A nucleic acid molecule, which is antisense to the nucleic acid encoding the progranulin, may be used as an inhibitor. The ‘antisense’ nucleic acid includes a nucleic acid sequence complementary to the ‘sense’ nucleic acid encoding the progranulin, for example, a nucleic acid sequence complementary to the coding strand of the double-stranded cDNA molecule or to the mRNA sequence. Thus, the antisense nucleic acid may form a hydrogen bond with the sense nucleic acid. The antisense nucleic acid may be complementary to all of the progranulin coding strands or a portion (e.g. the coding region) thereof. The antisense nucleic acid molecule may be complementary to the entire coding region of progranulin mRNA, but preferably useful is an oligonucleotide that is antisense to only a portion (e.g. a translation initiator) of the coding or noncoding region of progranulin mRNA. The antisense oligonucleotide may have a length of, for example, about 5 to 50 nucleotides. The antisense nucleic acid may be constructed through chemical synthesis and enzymatic binding reaction using known methods.

3) Peptide Mimetics

Binding an original progranulin polypeptide to VHL may be suppressed by manufacturing mimetics (e.g. peptide or non-peptide drug) in which the protein-binding domain of the progranulin polypeptide is inhibited.

The composition may further contain at least one active ingredient that exhibits the same or similar function, in addition to the progranulin inhibitor.

The composition may contain 0.1 to 90 parts by weight of progranulin based on 100 parts by weight of the composition.

The composition may be orally or parenterally administered upon clinical administration. Upon parenteral administration, the composition may be administered through intraperitoneal injection, intrarectal injection, subcutaneous injection, intravenous injection, intramuscular injection, epidural injection in the uterus, cerebral vascular injection or intrathoracic injection, and may be provided in the form of a typical medical formulation.

The composition may be used alone or in combination with surgery, radiation therapy, hormonal therapy, chemotherapy, and methods using biological response modifiers.

The composition is administered daily in an amount of about 0.0001 to 100 mg/kg, and preferably 0.001 to 10 mg/kg, and is preferably administered once or divided into multiple administrations several times per day, but the amount thereof may vary depending on the patient's body weight, age, gender, state of health, diet, administration time, administration method, excretion rate, and severity of disease.

The composition may be administered in the form of any parenteral formulation upon real-world critical use, and may be formulated with the typical use of an excipient or a diluent such as a filler, an extender, a binder, a humectant, a disintegrant, a surfactant, etc. The formulation for parenteral administration includes a sterile aqueous solution, a non-aqueous solvent, a suspension, an emulsion, a lyophilized preparation, or a suppository. The non-aqueous solvent or suspension may include propylene glycol, polyethylene glycol, a vegetable oil such as olive oil, an injectable ester such as ethyloleate, etc. The substrate for a suppository includes Witepsol, Macrogol, Tween 61, cacao butter, laurin, glycerogelatin, etc.

In addition, the present invention addresses a kit for the diagnosis, treatment or prognostic evaluation of osteoporosis, including any one selected from the group consisting of a nucleic acid complementary to a progranulin gene, a primer or probe specific to a progranulin gene, and an antibody binding to a progranulin protein.

In addition, the present invention addresses a method of measuring the expression level of progranulin for providing information about the diagnosis, treatment or prognostic evaluation of osteoporosis, comprising: 1) measuring the mRNA or protein expression level of progranulin in the blood, plasma or serum separated from a subject individual; and 2) screening the subject individual in which the mRNA or protein expression level of progranulin is increased compared to a normal control.

In this method, the mRNA expression level of progranulin may be measured using any one process selected from the group consisting of reverse transcription polymerase chain reaction (RT-PCR), quantitative or semi-quantitative RT-PCR, quantitative or semi-quantitative real-time RT-PCR, northern blot, and DNA or RNA chip.

In this method, the protein expression level of progranulin may be measured using any one process selected from the group consisting of tissue immunostaining, enzyme immunoassay (ELISA), and western blot.

Also, the present invention addresses a method of screening an agent for the prevention or treatment of osteoporosis, comprising: 1) treating a progranulin expression cell line with a subject composition or compound; 2) measuring the mRNA or protein expression level of progranulin in the cell line; and 3) screening the subject composition or compound in which the mRNA or protein expression level of progranulin is decreased compared to a non-treated control.

In this method, the mRNA expression level of progranulin may be measured using any one process selected from the group consisting of RT-PCR, quantitative or semi-quantitative RT-PCR, quantitative or semi-quantitative real-time RT-PCR, northern blot, and DNA or RNA chip.

In this method, the protein expression level of progranulin may be measured using any one process selected from the group consisting of tissue immunostaining, enzyme immunoassay (ELISA), and western blot.

Also, the present invention addresses a composition for inducing the differentiation of osteoclasts, containing progranulin as an active ingredient.

Also, the present invention addresses a pharmaceutical composition for inhibiting bone metastasis of a prostate cancer cell line caused by differentiation of osteoclasts, containing a progranulin inhibitor as an active ingredient.

The progranulin is preferably composed of the amino acid sequence of SEQ ID NO: 1, in which any one or more amino acids of SEQ ID NO: 1 may be added, deleted or substituted, but the present invention is not limited thereto.

The progranulin inhibitor may be an expression or activation inhibitor. Specifically, the expression inhibitor of a progranulin protein is preferably any one selected from the group consisting of an antisense nucleotide, and RNAi [short interfering RNA, short hairpin RNA and micro RNA (miRNA)], each of which complementarily binds to mRNA of a progranulin gene, and the activation inhibitor of a progranulin protein is preferably any one selected from the group consisting of a compound, a peptide, peptide mimetics, an aptamer, and an antibody, each of which complementarily binds to a progranulin protein, but the present invention is not limited thereto.

The composition may further contain at least one active ingredient that manifests the same or similar function, in addition to the progranulin inhibitor.

The composition may contain 0.1 to 90 parts by weight of progranulin based on 100 parts by weight of the composition.

The composition may be orally or parenterally administered upon clinical administration. Upon parenteral administration, the composition may be administered through intraperitoneal injection, intrarectal injection, subcutaneous injection, intravenous injection, intramuscular injection, epidural injection in the uterus, cerebral vascular injection or intrathoracic injection, and may be provided in the form of a typical medical formulation.

The composition may be used alone or in combination with surgery, radiation therapy, hormonal therapy, chemotherapy and methods using biological response modifiers.

The composition is administered daily in an amount of about 0.0001 to 100 mg/kg, and preferably 0.001 to 10 mg/kg, and is preferably administered once or divided into multiple administrations several times per day, but the amount thereof may vary depending on the patient's body weight, age, gender, state of health, diet, administration time, administration method, excretion rate, and severity of disease.

The composition may be administered in the form of any parenteral formulation upon real-world critical use, and may be formulated with the typical use of an excipient or a diluent such as a filler, an extender, a binder, a humectant, a disintegrant, a surfactant, etc. The formulation for parenteral administration includes a sterile aqueous solution, a non-aqueous solvent, a suspension, an emulsion, a lyophilized preparation, or a suppository. The non-aqueous solvent or suspension may include propylene glycol, polyethylene glycol, a vegetable oil such as olive oil, an injectable ester such as ethyloleate, etc. The substrate for a suppository includes Witepsol, Macrogol, Tween 61, cacao butter, laurin, glycerogelatin, etc.

In addition, the present invention addresses a kit for the diagnosis, treatment or prognostic evaluation of bone metastasis of prostate cancer, including any one selected from the group consisting of a nucleic acid complementary to a progranulin gene, a primer or probe specific to a progranulin gene, and an antibody binding to a progranulin protein.

In addition, the present invention addresses a method of measuring the expression level of progranulin for providing information about the diagnosis, treatment or prognostic evaluation of bone metastasis of prostate cancer, comprising: 1) measuring the mRNA or protein expression level of progranulin in the blood, plasma or serum separated from a subject individual; and 2) screening the subject individual in which the mRNA or protein expression level of progranulin is increased compared to a normal control.

In this method, the mRNA expression level of progranulin may be measured using any one process selected from the group consisting of RT-PCR, quantitative or semi-quantitative RT-PCR, quantitative or semi-quantitative real-time RT-PCR, northern blot, and DNA or RNA chip.

In this method, the protein expression level of progranulin may be measured using any one process selected from the group consisting of tissue immunostaining, enzyme immunoassay (ELISA), and western blot.

Also, the present invention addresses a method of screening an agent for inhibiting bone metastasis of a prostate cancer cell line caused by differentiation of osteoclasts, comprising: 1) treating a progranulin expression cell line with a subject composition or compound; 2) measuring the mRNA or protein expression level of progranulin in the cell line; and 3) screening the subject composition or compound in which the mRNA or protein expression level of progranulin is decreased compared to a non-treated control.

In this method, the mRNA expression level of progranulin may be measured using any one process selected from the group consisting of RT-PCR, quantitative or semi-quantitative RT-PCR, quantitative or semi-quantitative real-time RT-PCR, northern blot, and DNA or RNA chip.

In this method, the protein expression level of progranulin may be measured using any one process selected from the group consisting of tissue immunostaining, enzyme immunoassay (ELISA), and western blot.

A better understanding of the present invention is given through the following examples.

Such examples are merely set forth to illustrate, but are not to be construed as limiting the scope of the present invention.

### EXAMPLE 1

### Evaluation of Promotion of Differentiation of Osteoclasts depending on Amount of Progranulin

Hematopoietic stem cells were sampled from mouse bone marrow and were then treated with M-CSF (50 ng/ml) and thus differentiated into macrophages. After checking the differentiation into macrophages, they were treated with RANKL (500 ng/ml) and simultaneously the effect of progranulin on the differentiation of osteoclasts (OC) in proportion to the increase in the amount thereof was evaluated. The acquirement of macrophages and the differentiation of osteoclasts were measured as follows. Specifically, in order to separate bone marrow cells, five-week-old male ICR mice (Damool Science (Daejeon, Korea)) were sacrificed through cervical dislocation, after which the femur and tibia were excised aseptically and the soft tissue was removed. Both ends of the ilium were cut, and the medullary cavity thereof was washed with water using a 1 mL syringe, thus obtaining bone marrow cells. The separated bone marrow cells were cultured for one day in an α-MEM (Gibco-BRL (Grand Island, NT, USA)) medium, containing 10% FBS (Gibco-BRL (Grand Island, NT, USA)) and 1% penicillin/streptomycin (Gibco-BRL (Grand Island, NT, USA)), and unattached cells were collected. The unattached cells, which are the progenitor cells of osteoclasts, were cultured for 3 days in an α-MEM medium, containing 10% FBS, 1% penicillin/streptomycin, and M-CSF (30 ng/mL) (Peprotech (London, UK)). After three days, testing was performed using the attached macrophages (bone marrow macrophages, BMM). While the macrophages were treated with M-CSF (30 ng/mL) and RANKL (100 ng/mL) (Peprotech (London, UK)) and cultured, progranulin (AdipoGen (Incheon, Korea)) was added at different concentrations of 50, 250, 500, 1000 and 2000 ng/mL. Four days after the induction of differentiation, the cultured cells were stained with a TRAP solution (Sigma Aldrich, St. Louis, Mo., USA), and the red-stained cells (mature osteoclasts) were counted to analyze the extent of differentiation. As such, the TRAP⁺ stained cells of the multinucleated cells containing three or more nuclei per cell were counted.

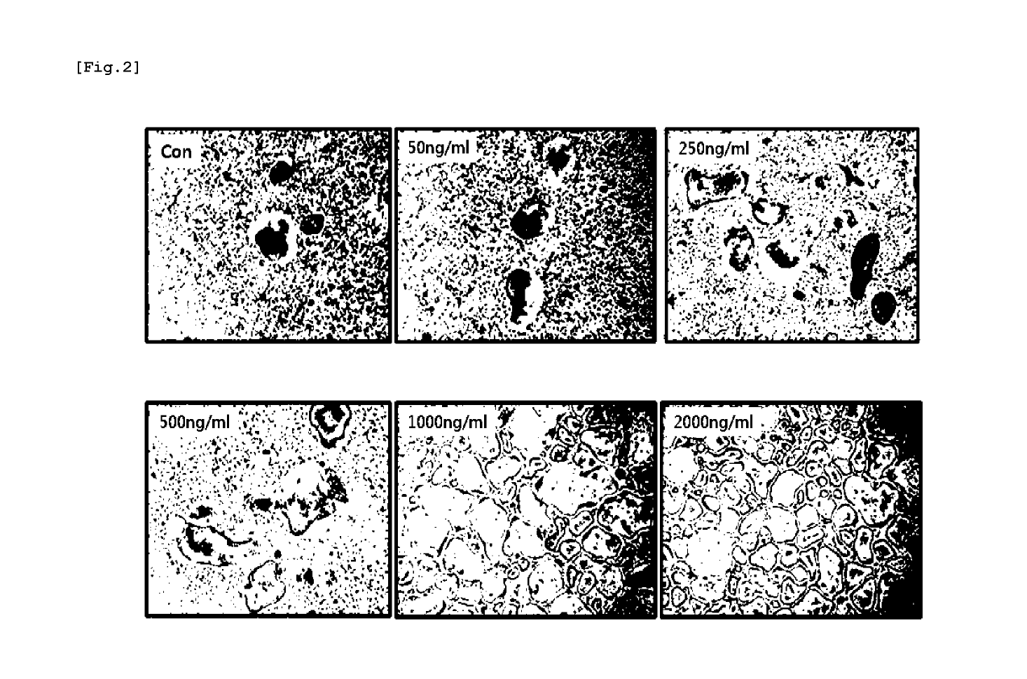

Consequently, as illustrated in FIG. 2, the differentiation of osteoclasts was remarkably promoted in proportion to an increase in the concentration of progranulin (FIG. 2).

### EXAMPLE 2

### Suppression of Differentiation of Osteoclasts by Progranulin Inhibition

In the case where the expression of progranulin underwent knockdown (KD) in the macrophages of <Example 1>, the effect on the differentiation of osteoclasts by RANKL was evaluated. The transfection of shRNA and the differentiation of osteoclasts were measured as follows. Specifically, shRNA retroviral packaging was performed by introducing shRNA (Transomic Technologies, Inc. (Huntsville, Ala.)) to Plat E cells using X-tremeGENE 9 (Roche, Nutley, N.J., USA). 48 hr after the transfection, the viral supernatant was recovered from the cultured solution and was then aliquoted into BMM together with Polybrene (8 g/mL) and thus cultured. After infection, the cells were cultured for 24 hr, separated using StemPro Accutase Cell Dissociation Reagent (Invitrogen), and further cultured for 2 days with the addition of M-CSF (30 ng/mL) and puromycin (2 g/mL). The BMM resistant to puromycin was treated with M-CSF (30 ng/mL) and RANKL (100 ng/mL), cultured for 4 days, and stained with a TRAP solution to analyze the extent of differentiation. The efficiency of transfection of shRNA was measured through reverse transcription polymerase chain reaction (RT-PCR). The total RNA in the cells was extracted based on the instruction using QIAzol lysis reagent (QIAGEN, Valencia, Calif., USA). The same amount of RNA was synthesized into cDNA using a TOPscript™ cDNA synthesis kit (Enzynomics, Daejeon, Korea). 1 g of cDNA was subjected to PCR using the following primers: progranulin (PGRN), Forward 5′-TTCACACACGATGCGTTTCA-3′ (SEQ ID NO: 2), Reverse 5′-AGGGCACACGACAGAAAAAG-3′ (SEQ ID NO: 3); GAPDH, Forward 5′-ACCACAGTCCATGCCATCAC-3′ (SEQ ID NO: 4), Reverse 5′-TCCACCACCCTGTTGCTGTA-3′ (SEQ ID NO: 5). The PCR was amplified in such a manner that denaturation at 94° C. for 30 sec, annealing at 58° C. for 30 sec, and extension at 72° C. for 30 sec were performed for 25 to 30 cycles. The PCR product was separated from 1% agarose gel, stained with EtBr, and observed at a UV wavelength.

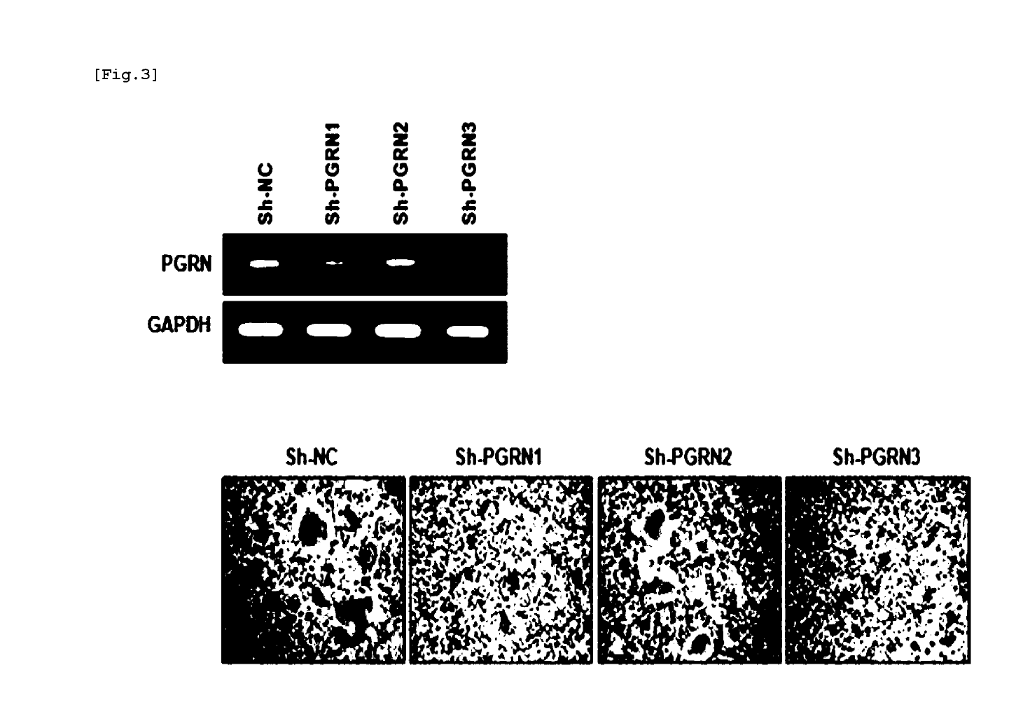

Consequently, as illustrated in FIG. 3, when the expression of progranulin was suppressed with three knockdown shRNA-retroviruses, the differentiation of osteoclasts by RANKL was significantly inhibited (FIG. 3).

### EXAMPLE 3

### Evaluation of Expression level of Progranulin in Osteoporosis Mouse Model

LPS (20 μg/ml) was intraperitoneally administered to the mice. After three to four days, osteoporosis was observed, and the serum of each mouse was sampled to thus evaluate changes in the expression of serum PGRN with mouse PGRN ELISA. The serum separation and progranulin ELISA were performed as follows. Specifically, five-week-old male ICR mice were divided into a control (a saline treated group) and a test group (an LPS treated group; 5 mg/kg), and were subjected to intraperitoneal injection for 8 days, after which blood was taken therefrom. Also, human blood was divided into a normal group (normal: 5 persons) and patient groups (prostate cancer: 8 persons, osteoporosis: 10 persons, bedridden: 4 persons). Each blood was centrifuged at 3,000 rpm for 15 min and only the serum was collected. It was compared with a standard material in accordance with the instruction using the mouse progranulin ELISA kit (AdipoGen, Incheon, Korea), and measured and analyzed at 450 nm using an ELISA reader (Bio-Tec instruments Inc., USA).

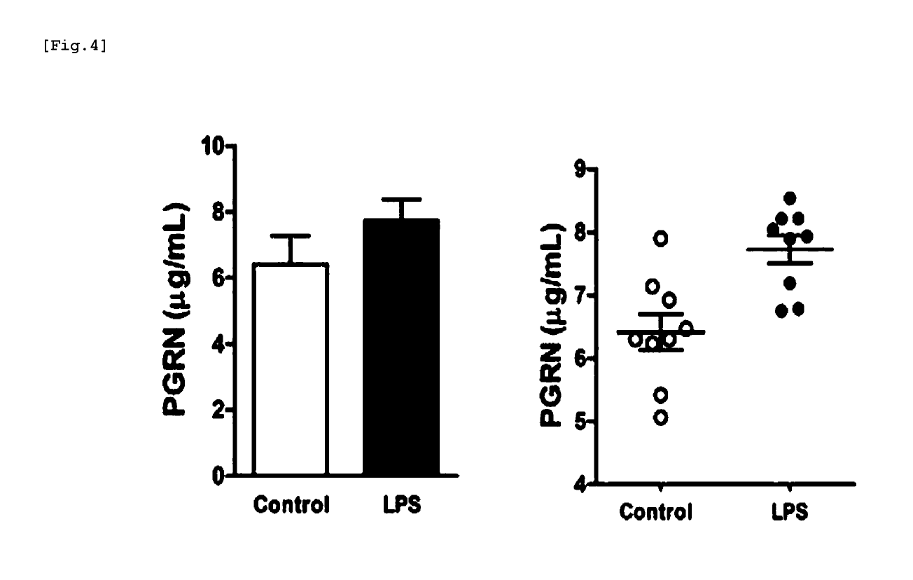

Consequently, as illustrated in FIG. 4, a statistically significant increase in the expression of progranulin was observed in the LPS-treated mouse serum compared to the mouse control (FIG. 4). Therefore, the progranulin was determined to be a factor that inhibits the osteoclast differentiation induced by LPS, rather than the progranulin being a simple product of LPS activation.

### EXAMPLE 4

### Evaluation of Expression Level of Progranulin in Blood derived from Human Osteoporosis, Prostate Cancer, and Bedridden Patients

Whether progranulin was increased in the serum of human osteoporosis patients and bedridden patients with low bone mineral density was observed.

Specifically, serum separation and progranulin ELISA were performed as follows. Human blood was divided into a normal group (normal: 5 persons) and patient groups (prostate cancer: 8 persons, osteoporosis: 10 persons, bedridden: 4 persons). As such, the normal group contained five healthy males and one healthy female, and the blood of patients was collected from patients who visited the Wonkwang University Hospital. The normal and patient groups were as follows: 1) normal group: (male, 50 years), (male, 43 years), (male, 29 years), (male, 28 years), (male, 24 years), (female, 30 years), 2) prostate cancer patient group: (male, 75 years), (male, 72 years), (male, 71 years), (male, 72 years), (male, 72 years), (male, 77 years), (male, 77 years), (male, 77 years), (male, 79 years), 3) osteoporosis patient group: (female, 55 years), (female, 60 years), (female, 51 years), (female, 48 years), (female, years), (female, 59 years), (female, 49 years), (female, 72 years), (female, 75 years), (female, 52 years), (female, 58 years), 4) bedridden patient group: (female, 50 years), (male, 61 years), (male, 54 years), (male, 76 years).

TABLE 1

| | Classification | Patient No. | Age | Gender | Progranulin (ng/mL) |
|---|---|---|---|---|---|
| 1 | Normal | | 50 | Male | 72.089 |
| 2 | Normal | | 43 | Male | 94.640 |
| 3 | Normal | | 31 | Female | 60.259 |
| 4 | Normal | | 29 | Male | 66.174 |
| 5 | Normal | | 28 | Male | 68.392 |
| 6 | Normal | | 24 | Male | 56.932 |
| 1 | Prostate Cancer | 302460 | 75 | Male | 161.183 |
| 2 | Prostate Cancer | 983283 | 72 | Male | 79.852 |
| 3 | Prostate Cancer | 798203 | 71 | Male | 144.917 |
| 4 | Prostate Cancer | 657047 | 72 | Male | 144.177 |
| 5 | Prostate Cancer | 747710 | 72 | Male | 213.309 |
| 6 | Prostate Cancer | 869154 | 77 | Male | 129.390 |
| 7 | Prostate Cancer | 1087655 | 77 | Male | 100.185 |
| 8 | Prostate Cancer | 142370 | 77 | Male | 157.486 |
| 9 | Prostate Cancer | 708704 | 70 | Male | 94.270 |
| 1 | Osteoporosis | 1119164 | 55 | Female | 117.930 |
| 2 | Osteoporosis | 486692 | 60 | Female | 96.118 |
| 3 | Osteoporosis | 1109653 | 51 | Female | 65.434 |
| 4 | Osteoporosis | 1014188 | 48 | Female | 110.536 |
| 5 | Osteoporosis | 1086537 | 52 | Female | 121.257 |
| 6 | Osteoporosis | 489097 | 59 | Female | 96.118 |
| 7 | Osteoporosis | 958843 | 49 | Female | 131.238 |
| 8 | Osteoporosis | 1054401 | 72 | Female | 153.050 |
| 9 | Osteoporosis | 781400 | 75 | Female | 134.196 |
| 10 | Osteoporosis | 894009 | 52 | Female | 128.651 |
| 11 | Osteoporosis | 346218 | 58 | Female | 132.348 |
| 1 | Bedridden | 308760 | 50 | Female | 151.941 |
| 2 | Bedridden | 1116522 | 61 | Male | 91.682 |
| 3 | Bedridden | 819060 | 54 | Male | 62.847 |
| 4 | Bedridden | 1111566 | 76 | Male | 141.220 |

Each blood was centrifuged at 3,000 rpm for 15 min to isolate the serum. It was compared with a standard material in accordance with the instruction using the human progranulin ELISA kit (AdipoGen, Incheon, Korea), and measured and analyzed at 450 nm using an ELISA reader (Bio-Tec instruments Inc., USA).

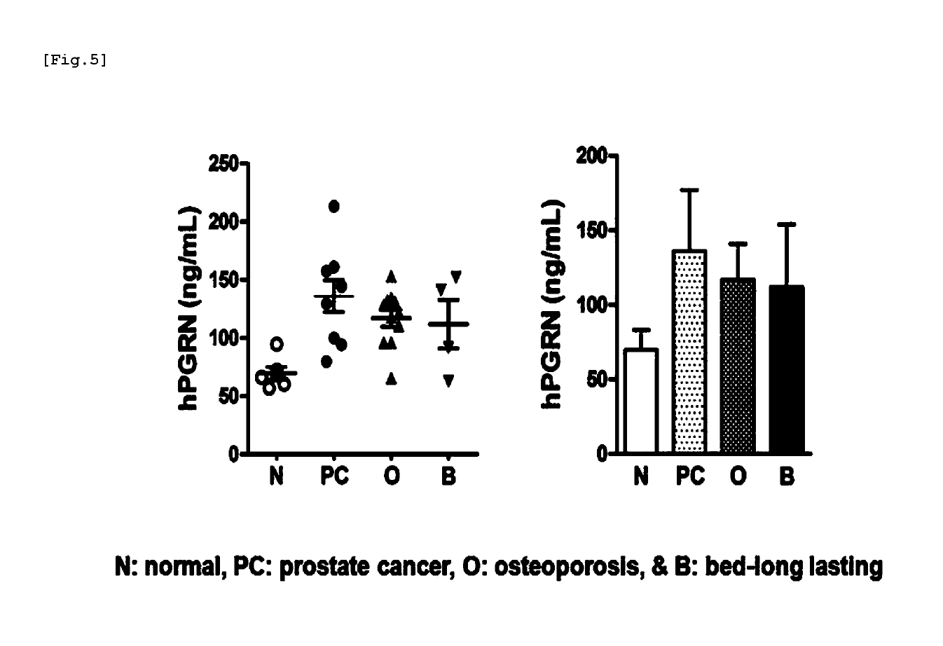

Consequently, as illustrated in FIG. 5, higher serum progranulin concentrations were detected in the prostate cancer patients, osteoporosis patients, and bedridden patients with low bone mineral density than in the normal group (FIG. 5). These test results were matched with the results of in-vitro and in-vivo testing with mice.

### INDUSTRIAL APPLICABILITY

According to the present invention, progranulin functions as a RANK-dependent cell-communication factor or as a mediator in osteoporosis through LPS-induced inflammation, and the expression level thereof is high in the serum of prostate cancer patients, osteoporosis patients, and bedridden patients with low bone mineral density, compared to a control. Therefore, the use of progranulin as a biomarker is effective at developing an agent for inhibiting bone metastasis of prostate cancer cells or an osteoporosis therapeutic agent.

[Sequence Listing Free Text]

SEQ ID NO: 1 is an amino acid sequence of human progranulin.

SEQ ID NO: 2 is a base sequence of a primer used for RT-PCR to analyze the efficiency of transfection of shRNA in an example of the present invention, that is, a base sequence of a forward primer for progranulin (PGRN).

SEQ ID NO: 3 is a base sequence of a primer used for RT-PCR to analyze the efficiency of transfection of shRNA in an example of the present invention, that is, a base sequence of a reverse primer for progranulin.

SEQ ID NO: 4 is a base sequence of a primer used for RT-PCR to analyze the efficiency of transfection of shRNA in an example of the present invention, that is, a base sequence of a forward primer for GAPDH.

SEQ ID NO: 5 is a base sequence of a primer used for RT-PCR to analyze the efficiency of transfection of shRNA in an example of the present invention, that is, a base sequence of a reverse primer for GAPDH.

1. A method of preventing or treating osteoporosis in a subject, comprising:
providing a pharmaceutical composition comprising a therapeutically effective amount of a progranulin inhibitor as an active ingredient and
administering the pharmaceutical composition to the subject, wherein the osteoporosis is prevented or treated.

2. The method of claim 1, wherein the progranulin comprises an amino acid sequence of SEQ ID NO: 1.

3. The method of claim 1, wherein the progranulin inhibitor comprises any one selected from the group consisting of an antisense nucleotide, short interfering RNA, and short hairpin RNA, each of which complementarily binds to mRNA of a progranulin gene, and an aptamer and an antibody, each of which binds to a progranulin protein.

4. A for method of inhibiting differentiation of an osteoclast in a subject, comprising:
providing a pharmaceutical composition comprising a therapeutically effective amount of a progranulin inhibitor as an active ingredient and
administering the pharmaceutical composition to the subject, wherein the differentiation of the osteoclast is inhibited.

5. A method of determining a diagnosis of osteoporosis, comprising:
measuring an mRNA or protein expression level of progranulin in blood, plasma or serum separated from a subject; and
comparing the subject in which the mRNA or protein expression level of progranulin to a normal control; and
diagnosing the osteoporosis if the mRNA or protein expression level of progranulin in the subject is increased compared to the normal control.

6. The method of claim 5, wherein the mRNA expression level of progranulin is measured by any one process selected from the group consisting of reverse transcription polymerase chain reaction (RT-PCR), quantitative or semi-quantitative RT-PCR, quantitative or semi-quantitative real-time RT-PCR, northern blot, and DNA or RNA chip.

7. The method of claim 5, wherein the protein expression level of progranulin is measured by any one process selected from the group consisting of tissue immunostaining, enzyme immunoassay (ELISA), and western blot.

8. A kit for diagnosis, treatment or prognostic evaluation of osteoporosis, comprising: at least one selected from the group consisting of a nucleic acid complementary to a progranulin gene, a primer or probe specific to a progranulin gene, and an antibody binding to a progranulin protein.

9. A method of screening an agent for prevention or treatment of osteoporosis, comprising:
1) treating a progranulin expression cell line with a subject composition or compound;
2) measuring an mRNA or protein expression level of progranulin in the cell line; and
3) screening the subject composition or compound in which the mRNA or protein expression level of progranulin is decreased compared to a non-treated control.

10. A method of screening an agent for inhibiting differentiation of an osteoclast, comprising:
1) treating a progranulin expression cell line with a subject composition or compound;
2) measuring an mRNA or protein expression level of progranulin in the cell line; and
3) screening the subject composition or compound in which the mRNA or protein expression level of progranulin is decreased compared to a non-treated control.

11. (canceled)

12. A method for inhibiting bone metastasis of a prostate cancer cell line, comprising:
administering to a subject a pharmaceutical composition containing a progranulin inhibitor as an active ingredient, wherein the bone metastais of the prostate cell line caused by differentiation of the osteoclast is inhibited.

13. The method of claim 12, wherein the progranulin comprises an amino acid sequence of SEQ ID NO: 1.

14. The method of claim 12, wherein the progranulin inhibitor comprises any one selected from the group consisting of an antisense nucleotide, short interfering RNA, and short hairpin RNA, each of which complementarily binds to mRNA of a progranulin gene, and an aptamer and an antibody, each of which binds to a progranulin protein.

15. A method of measuring an expression level of progranulin for providing information about diagnosis, treatment or prognostic evaluation of bone metastasis of prostate cancer, comprising:
1) measuring an mRNA or protein expression level of progranulin in blood, plasma or serum separated from a subject individual; and
2) screening the subject individual in which the mRNA or protein expression level of progranulin is increased compared to a normal control.

16. The method of claim 15, wherein the mRNA expression level of progranulin is measured using any one process selected from the group consisting of RT-PCR, quantitative or semi-quantitative RT-PCR, quantitative or semi-quantitative real-time RT-PCR, northern blot, and DNA or RNA chip.

17. The method of claim 15, wherein the protein expression level of progranulin is measured using any one process selected from the group consisting of tissue immunostaining, enzyme immunoassay (ELISA), and western blot.

18. A kit for diagnosis, treatment or prognostic evaluation of bone metastasis of prostate cancer, comprising:
at least one selected from the group consisting of a nucleic acid complementary to a progranulin gene, a primer or probe specific to a progranulin gene, and an antibody binding to a progranulin protein.

19. A method of screening an agent for inhibiting bone metastasis of a prostate cancer cell line caused by differentiation of an osteoclast, comprising:
1) treating a progranulin expression cell line with a subject composition or compound;
2) measuring an mRNA or protein expression level of progranulin in the cell line; and
3) screening the subject composition or compound in which the mRNA or protein expression level of progranulin is decreased compared to a non-treated control.

20. The method of claim 19, wherein the mRNA expression level of progranulin is measured using any one process selected from the group consisting of RT-PCR, quantitative or semi-quantitative RT-PCR, quantitative or semi-quantitative real-time RT-PCR, northern blot, and DNA or RNA chip.

21. The method of claim 19, wherein the protein expression level of progranulin is measured using any one process selected from the group consisting of tissue immunostaining, enzyme immunoassay (ELISA), and western blot.
